(19) United States
(12) Patent Application Publication
(10) Pub. No.: US 2025/0264429 A1
(43) Pub. Date: Aug. 21, 2025

(54) SYSTEME DE DETERMINATION D'UNE QUANTITE D'EMISSIONS DE DIOXYDE DE CARBONE RESULTANT DE L'ECHAUFFEMENT D'UN CONDUCTEUR ELECTRIQUE D'UN CABLE ELECTRIQUE PAR EFFET JOULE

(71) Applicant: NEXANS, COURBEVOIE (FR)

(72) Inventor: Raphaël GUFFOND, LYON (FR)

(21) Appl. No.: 19/060,298

(22) Filed: Feb. 21, 2025

(30) Foreign Application Priority Data
Feb. 21, 2024 (FR) FR2401716

(51) Int. Cl. G01N 27/18

(52) U.S. Cl. CPC G01N 27/18

(57) ABSTRACT

A system for determining a quantity of emissions of carbon dioxide resulting from the heating of an electrical conductor of an electric cable by the Joule effect, said determination system includes an electrical cable including at least one electrical conductor and at least one layer of material surrounding said at least one conductor, a measuring unit associated with the electrical cable, said measuring unit including at least one temperature sensor, a calculation unit being configured to communicate information with the measurement unit, the calculation unit being configured to determine the conductor temperature Θ_{cond} by means of said at least one temperature sensor, said calculation unit being further configured to determine an quantity of emissions of carbon dioxide resulting from the heating of the electrical conductor by the Joule effect as a function of the conductor temperature Θ_{cond}.

### TECHNICAL FIELD

The present invention concerns a system for determination of a quantity of emissions of carbon dioxide resulting from the heating of an electrical conductor of an electrical cable by the Joule effect.

The invention more specifically concerns a non-invasive determination system.

### TECHNOLOGICAL BACKGROUND

In an electrical distribution network, heating of the electrical conductor of an electrical cable is produced by the Joule effect when a current flows in the electrical conductor. With this increase in temperature, the resistance of the electrical conductor increases, which leads to an increase in the power loss.

This annual power loss directly impacts the quantity of emissions of carbon dioxide (CO₂).

At present there exist no integrated or add-on solutions for determination of these emissions of carbon dioxide resulting from the heating of an electrical conductor of an electrical cable by the Joule effect.

There is therefore a need for a system for determination of a quantity of emissions of carbon dioxide resulting from the heating of an electrical conductor of an electric cable by the Joule effect directly integrated into an electric cable or added thereto.

### SUMMARY OF THE INVENTION

To this end, the invention provides a system for determination of a quantity of emissions of carbon dioxide resulting from the heating of an electrical conductor of an electrical cable by the Joule effect, said determination system comprising:
an electrical cable comprising at least one electrical conductor and at least one layer of material surrounding said at least one conductor,
a measurement unit associated with the electrical cable, said measurement unit comprising at least one temperature sensor,
a calculation unit being configured to communicate information with the measurement unit, the calculation unit being configured to determine the conductor temperature Θ_{cond} by means of said at least one temperature sensor, said calculation unit being further configured to determine an quantity of emissions of carbon dioxide resulting from the heating of the electrical conductor by the Joule effect as a function of the conductor temperature Θ_{cond}.

It has appeared that the measurement of temperature is a particularly simple and advantageous means for determining the amount of CO₂ emissions induced by heating of the electrical conductor. The measurement of temperatures makes it possible, for example, to determine a heat flux (or thermal power) from which the emissions of CO₂ can be deduced.

Preferably, the calculation unit is configured to determine an electrical intensity I_{cond} of the electrical conductor as a function of the conductor temperature Θ_{cond}. The configuration of the calculation unit for determination of the electric intensity I_{cond} from the conductor temperature Θ_{cond} makes it possible to obtain a measurement system that is particularly simple to implement and inexpensive since it does not comprise any sensor other than the temperature sensor(s).

In accordance with one embodiment of the determination system, the calculation unit is configured to determine an electrical resistance R_{c} of the electrical conductor as a function of the conductor temperature Θ_{cond}.

This electrical resistance R_{c} is in particular determined as follows:

$${R}_{c} = {R}_{0} \times ( 1 + {α}_{20} \times ( {Θ}_{cond} - 20 ) ) \times ( 1 + {y}_{s} + {y}_{p} )$$

In accordance with one embodiment of the determination system, the calculation unit is configured to determine a power loss PₒL as a function of the electrical intensity I_{cond} in the electrical conductor and the electrical resistance R_{c} of the electrical conductor, the calculation unit being configured to determine the quantity of emissions of carbon dioxide as a function of said power loss PₒL.

This power loss PₒL is in particular determined as follows:

$$PoL = Rc \times {I}_{cond}^{2}$$

In accordance with one embodiment of the determination system, the latter further comprises a measurement module mounted on the electrical cable, said measurement module comprising at least the temperature sensor.

In accordance with one embodiment of the determination system, the measurement module further comprises the calculation unit.

In accordance with one embodiment of the determination system, the measurement module is configured to be removably mounted on the electrical cable.

In accordance with one embodiment of the determination system, the measurement module comprises a device for fixing it to the electrical cable.

In accordance with one embodiment of the determination system, said at least one temperature sensor comprises a temperature sensor that is disposed on an external surface of said at least one layer of material to measure a peripheral temperature Θ_{b1} at the level of the external surface of said at least one layer of material, the calculation unit being configured to determine the conductor temperature Θ_{cond} as a function of the peripheral temperature Θ_{b1}.

The use of one or more temperature sensors external to the electrical cable enables determination of the conductor temperature Θ_{cond} in a non-invasive and non-destructive manner.

In accordance with one embodiment of the determination system, the latter further comprises:
an additional layer of material disposed around said at least one layer of material and covering said at least one temperature sensor,
at least one additional temperature sensor disposed on an external surface of said layer of additional material to measure an additional peripheral temperature Θ_{b2} at the level of the external surface of said at least one additional layer of material.

In accordance with one embodiment of the determination system, the calculation unit is configured to determine the conductor temperature Θ_{cond} as a function of the peripheral temperature Θ_{b1} and the additional peripheral temperature Θ_{b2}.

Said at least one layer of material of the electric cable has a layer thermal resistance T₁.

The calculation unit may comprise a unit for determination of the conductor temperature Θ_{cond}. Thus, the determination unit is configured to determine the conductor temperature Θ_{cond} as a function of the measured peripheral temperature Θ_{b1}, the layer thermal resistance T₁ and the thermal flux We generated by the circulation of an electric current in the electrical conductor.

The conductor temperature Θ_{cond} is determined here by means of a physical model using the measured peripheral temperature Θ_{b1}, and the layer thermal resistance T₁ and the heat flux W_{c} generated by the circulation of an electric current in the electrical conductor.

The electrical intensity I_{cond} is determined by means of the temperature value of the conductor Θ_{cond} and knowledge of the geometry of the associated conductor.

The use of this physical model makes it possible to dispense with the utilisation of a temperature internal to the electrical cable, i.e. measured via a component disposed in the proximity of the conductor, or more generally inside the external sheath of the electrical cable.

The physical model used enables estimation of the conductor temperature Θ_{cond} by means of the measured peripheral temperature Θ_{b1}, and the layer thermal resistance T₁ and the heat flux W_{c} generated by the circulation of an electric current in the electrical conductor.

In accordance with one embodiment of the determination system, the determination system further comprises:
at least one additional layer of material disposed around said at least one layer of material and covering said at least one temperature sensor, said additional layer of material having an additional thermal resistance T_{b},
at least one additional temperature sensor disposed on an external surface of said additional layer of material to measure an additional peripheral temperature Θ_{b2} at the level of the external surface of said at least one additional layer of material.

In accordance with one embodiment of the determination system, said additional layer of material extends around said at least one layer of material over only a portion of the length of the electrical cable.

In accordance with one embodiment of the determination system, the at least one additional layer of material comprises:
a first additional layer portion having a first additional thermal resistance T_{b}, and
a second additional layer portion having a second additional thermal resistance T′_{b}, the first T_{b} and second T′_{b} additional thermal resistances being different,
   and in which said at least one additional temperature sensor comprises:
at least one first additional temperature sensor disposed on an external surface of the first additional layer portion to measure a first additional peripheral temperature Θ_{b2},
at least one second additional temperature sensor disposed on an external surface of the second additional layer portion to measure a second additional peripheral temperature Θ′_{b2},
the determination unit being configured to determine the conductor temperature Θ_{cond} as a function of the first T_{b} and the second T′_{b} additional thermal resistances as well as the first Θ_{b2} and the second Θ′_{b2} additional peripheral temperatures.

In accordance with one embodiment of the determination system, said at least one first additional layer and said at least one second additional layer are disposed on different angular sectors around the electrical conductor.

Said at least one first additional layer and said at least one second additional layer may overlap at least partially.

In accordance with one embodiment of the determination system, said at least one first additional layer and said at least one second additional layer together form a layer extending continuously around the electrical conductor in a plane perpendicular to the longitudinal axis along which the electrical conductor extends.

In accordance with one embodiment of the determination system, said at least one temperature sensor comprises:
at least one first sensor disposed on the external surface of said at least one layer of material, between said external surface and the first additional layer portion, to measure a peripheral temperature Θ_{b1} at the level of the external surface of said at least one layer of material,
at least one second sensor disposed on the external surface of said at least one layer of material, between said external surface and the second additional layer portion, to measure a peripheral temperature Θ′_{b1} at the level of the external surface of said at least one layer of material.

In accordance with one embodiment of the determination system, the determination unit is configured to determine the conductor temperature Θ_{cond} on the basis of the following equation:

$${θ}_{conductor} = \frac{( {θ}_{b 1} {T}_{b} - {θ}_{b 1}^{′} {T}_{b}^{′} ) \times ( ( {θ}_{b 1} - {θ}_{b 2} ) )}{{T}_{b} ( {θ}_{b 1}^{′} - {θ}_{b 2}^{′} ) - {T}_{b}^{′} ( {θ}_{b 1} - {θ}_{b 2} )}$$

T_{b} being the first additional thermal resistance of the first additional layer of material,
T′_{b} being the second additional thermal resistance of the second additional layer of material,
Θ_{b1} is the peripheral temperature measured by said at least one first temperature sensor,
Θ_{b2} is the additional peripheral temperature measured by said at least one first additional temperature sensor,
Θ′_{b1} is the peripheral temperature measured by said at least one second temperature sensor,
Θ′_{b2} is the additional peripheral temperature measured by said at least one second additional temperature sensor.

In accordance with one embodiment of the determination system, the first and second additional layer portions are made of a different material.

In accordance with one embodiment of the determination system, the first and second additional layer portions have a different thickness perpendicular to a longitudinal axis along which the electrical cable extends.

In accordance with one embodiment of the determination system, said at least one layer of material comprises an external sheath forming said external surface, said at least one temperature sensor being disposed on said external sheath.

In accordance with one embodiment of the determination system, the determination unit is configured to determine a conductor temperature Θ_{cond} as a function of the measured peripheral temperature Θ_{b1} and the layer thermal resistance T₁ and the thermal flux W_{c} generated by the circulation of an electrical current in the electrical conductor.

In accordance with one embodiment of the determination system, the determination unit is configured to determine the conductor temperature Θ_{cond} on the basis of the following equation:

$${θ}_{cond} = {θ}_{b 1} + {W}_{c} * {T}_{1}$$

The determination unit can be disposed in the proximity of the electric cable or at a distance from the latter.

In accordance with one embodiment of the determination system, said at least one layer of material comprises an external sheath forming said external surface, said at least one temperature sensor being disposed on said external sheath.

In accordance with one embodiment of the determination system, the latter further comprises:
an additional layer of material disposed around said at least one layer of material and covering said at least one temperature sensor, said additional layer of material having an additional thermal resistance Tb
at least one additional temperature sensor disposed on an external surface of said layer of additional material to measure an additional peripheral temperature Θb2 at the level of the external surface of said at least one additional layer of material.

In accordance with one embodiment of the determination system, said additional layer of material extends around said at least one layer of material over only a portion of the length of the electrical cable.

In accordance with one embodiment of the determination system, the determination unit is configured to determine the conductor temperature Θ_{cond} on the basis of the following equation:

$${θ}_{cond} = {θ}_{b 1} + \frac{( {θ}_{b 1} - {θ}_{b 2} )}{{T}_{b}} \times {T}_{1}$$

Θ_{b1} is the peripheral temperature measured by said at least one temperature sensor,
Θ_{b2} is the additional peripheral temperature measured by said at least one additional temperature sensor,
T_{b} being the additional thermal resistance of the additional layer of material,
T₁ being the thermal resistance of said at least one layer of material.

In accordance with one embodiment of the determination system, the determination unit is configured to determine the conductor temperature Θ_{cond} as a function of a heating W_{d} from a dielectric loss in the at least one layer of material.

In accordance with one embodiment of the determination system, the determination unit is configured to determine the conductor temperature Θ_{cond} on the basis of the following equation:

$${θ}_{cond} = {θ}_{b 1} + {T}_{1} \times ( {W}_{c} - \frac{{W}_{d}}{2} )$$

W_{d} being the heating from a dielectric loss in said at least one layer of material.

In accordance with one embodiment of the determination system, the determination unit is configured to determine the conductor temperature Θ_{cond} on the basis of the following equation:

$${θ}_{cond} = {θ}_{b 1} + {T}_{1} \times ( \frac{{θ}_{b 1} - {θ}_{b 2}}{{T}_{b}} - \frac{{W}_{d}}{2} )$$

W_{d} being the heating from a dielectric loss in said at least one layer of material.

In accordance with one embodiment of the determination system, said at least one additional layer of material and said at least one additional temperature sensor are carried by the measurement module.

This measurement module is preferably removable from the electrical cable in such a manner as to carry out a point and localised measurement on the electrical cable. This unit is for example a measurement accessory.

The measurement module preferably has a dimension along the longitudinal axis of the electrical cable such that it extends over only a portion of the length of the electrical cable.

### BRIEF DESCRIPTION OF THE FIGURES

The following description given with reference to the appended drawings, provided by way of non-limiting example, will clearly explain in what the invention consists and how it may be carried out. In the attached figures:

FIG. 1, represents a perspective view of a determination system comprising an electrical cable, a measurement unit, a calculation unit for determining a quantity of emissions of carbon dioxide resulting from the heating of an electrical conductor of the electrical cable;

FIG. 2 represents a perspective view of one embodiment of the determination system from FIG. 1 comprising a measurement module mounted on the electric cable, the measurement module comprising in particular the measurement unit;

FIG. 3 represents a view in section of the electrical cable in a first configuration comprising an electrical conductor and at least one layer of material;

FIG. 4 represents a view in section of the electrical cable from FIG. 3 comprising a plurality of sensors on an external surface of said at least one layer of material;

FIG. 5 represents a diagram of a first model of the electric cable;

FIG. 6 represents a view in section of the electrical cable of FIG. 3 in which the determination system comprises an additional layer of material around the electrical cable and a plurality of additional sensors disposed on an external surface of that additional layer of material;

FIG. 7 represents a diagram of a second model of the electrical cable;

FIG. 8 represents a third model of the electric cable in which the dielectric losses in said at least one layer of material are taken into account;

FIG. 9 represents a fourth model of the electrical cable in which the dielectric losses in said at least one layer of material are taken into account, according to the second mode of determination;

FIG. 10 represents a sectional view of the electrical cable in a second configuration comprising an electrical conductor, one or more layers of material surrounding the electrical conductor and a screen surrounding said layers of material;

FIG. 11 represents a fifth model of the electric cable in which the dielectric losses in said at least one layer of material are taken into account as well as the dielectric losses in the screen;

FIG. 12 represents a sixth model of the electrical cable to determine the conductor temperature Θ_{cond} in a manner independent of the surrounding environment;

FIG. 13 represents a sectional view of the electrical cable of FIG. 6 in which the determination system comprises an additional layer of material formed of first and second additional layer portions and a plurality of additional sensors disposed on an external surface of each of the first and second additional layer of material portions;

FIG. 14 represents a diagram of a seventh model of the electric cable using the first additional layer portion of FIG. 13;

FIG. 15 represents a diagram of the seventh model of the electrical cable using the second additional layer portion of FIG. 13;

FIG. 16 represents an equation for determining the conductor temperature according to the seventh model;

FIG. 17 represents a diagram of an eighth model of the electrical cable using the first additional layer portion of FIG. 13;

FIG. 18 represents a diagram of the eighth model of the electrical cable using the second additional layer portion of FIG. 13;

FIG. 19 represents an equation for determining the conductor temperature according to the eighth model.

### DESCRIPTION OF EMBODIMENT(S)

For reasons of clarity, the same references denoting the same elements according to the prior art and according to the invention are used for all the figures.

The concept of the invention is described more fully below with reference to the accompanying drawings, in which embodiments of the concept of the invention are shown. In the drawings, the size and the relative sizes of the elements may be exaggerated for reasons of clarity. Similar numbers refer to similar elements in all the drawings. However, this concept of the invention can be executed in numerous different forms and should not be interpreted as being limited to the embodiments discussed herein. Rather than that, these embodiments are proposed so that this description is complete, and communicate the extent of the concept of the invention to persons skilled in the art.

Any reference throughout the specification to “an embodiment” signifies that a functionality, a structure, or a particular feature described with reference to one embodiment is included in at least one embodiment of the present invention. Thus the occurrence of the expression “in one embodiment” at various places throughout the specification does not necessarily refer to the same embodiment. Furthermore, the functionalities, the structures or the particular features may be combined in any appropriate manner in one or more embodiments. Furthermore, the term “comprising” does not exclude other elements or steps.

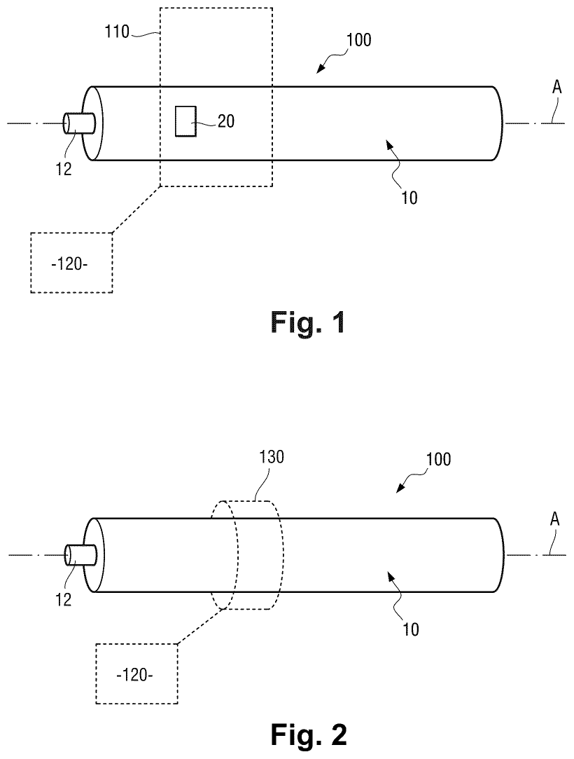

A system 100 for determination of a quantity of emissions of carbon dioxide resulting from the heating of an electrical conductor of an electrical cable by the Joule effect is depicted in FIG. 1.

This determination system 100 comprises an electric cable 10 comprising at least one electrical conductor 12 and at least one layer of material surrounding said at least one conductor. This layer of material is for example an insulative layer.

The electrical cable 10 extends along a longitudinal axis A.

This determination system 100 comprises a measurement unit 110 associated with the electrical cable 10. The measurement unit includes at least one temperature sensor 20.

The determination system 100 further comprises a calculation unit 120 configured to communicate information with the measurement unit 110.

The calculation unit 120 is configured to determine the conductor temperature Θ_{cond} and here also the electrical intensity I_{cond} of the electrical current circulating in the conductor 12 by means of said at least one temperature sensor.

The calculation unit 120 is configured to determine the electrical intensity I_{cond} as a function of the conductor temperature Θ_{cond} and the geometry of the conductor 12.

The IEC60287 standard enables to obtain the value of the resistance of an electrical conductor (R_{c}) from its temperature and from at least one element of the geometry of the conductor. The geometry element(s) comprises at least one of: the diameter of the conductor; the shape of the section of the conductor; the number of component(s) (wires for example) that make up the conductor; the diameter of these components; the shape of the section of the components; the way in which these components are disposed, etc.

For a given conductor, it is therefore possible to define a table, stored in a memory of the calculation unit, that associates resistance values R_{c} of the conductor 12 with values of the temperature Θ_{cond} of the conductor 12.

The measurement of temperatures in a material the thermal resistance of which is known enables to determine a heat transfer flux or thermal power representative of the power produced by the Joule effect. From P=R_{c}I_{cond}2, the intensity I_{cond} of the current circulating in the conductor 12 is calculated.

The calculation unit 120 is configured to determine the emissions of carbon dioxide. There are tables providing emissions of carbon dioxide as a function of thermal power. It is therefore possible to store in a memory of the calculation unit 120 such a table associating values of emissions of carbon dioxide with values of the thermal power given off by the conductor 12.

Preferably, said at least one temperature sensor 20 is disposed outside the electrical cable, i.e. on an external surface of this electrical cable 10. In this configuration, the conductor temperature Θ_{cond} is determined by means of a physical model described below in connection with FIGS. 4 to 12.

In a variant compatible with the invention, said at least one temperature sensor may be disposed inside the electrical cable 10. In this variant, the conductor temperature Θ_{cond} is measured directly in the proximity of the electrical conductor 12.

The calculation unit 120 is further configured to determine a quantity of emissions of carbon dioxide resulting from the heating of the electrical conductor by Joule effect as a function of the conductor temperature Θ_{cond} and of the electrical intensity I_{cond} in the electrical conductor.

Referring to FIG. 2, the determination system 100 may comprise a measurement module 130 in which are accommodated at least one of the at least one temperature sensor.

The entire measurement unit 110 is preferably carried by the measurement module 130.

The calculation unit 120 is configured to be in communication with the measurement unit 110. The calculation unit 120 can be dissociated from the measurement module 130 as illustrated in FIG. 2 or integrated into this measurement module 130.

This measurement module 130 is, for example, mounted in such a manner as to be removable from the electrical cable 10 order to produce a point and localised measurement on the electrical cable 10. This measurement module 130 is for example a portable measurement accessory.

The measurement module 130 has a dimension along the longitudinal axis A such that it extends over only a portion of the length of the electric cable 10. The measurement module 130 extends preferably around the electrical cable 10, i.e. about the longitudinal axis A.

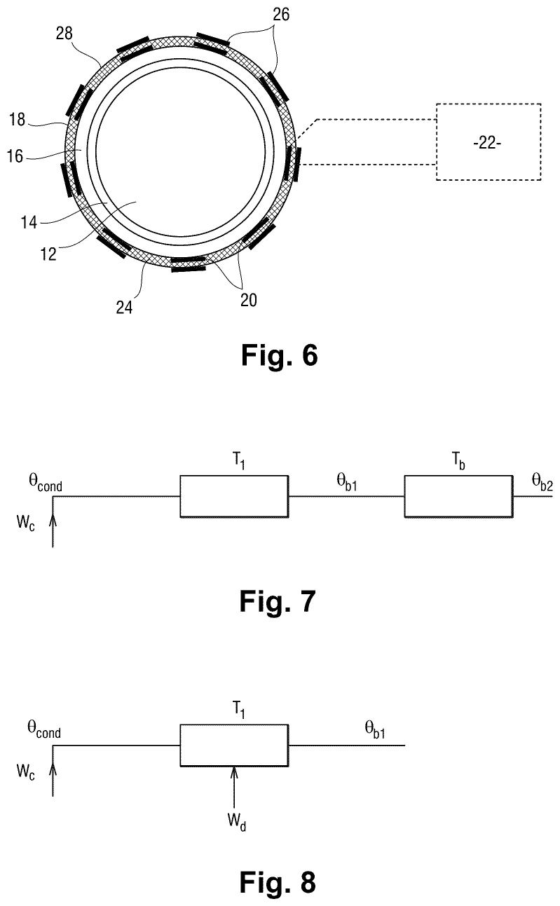

The measurement module 130 may also comprise an additional structure described hereinafter, notably with reference to FIGS. 6 and 10.

For the determination of the emissions of CO₂, the calculation unit 120 is configured to determine an electrical resistance R_{c} of the electrical conductor as a function of the conductor temperature Θ_{cond}.

This electrical resistance R_{c} is notably determined as follows:

$${R}_{c} = {R}_{0} \times ( 1 + {α}_{20} \times ( {Θ}_{cond} - 20 ) ) \times ( 1 + {y}_{s} + {y}_{p} )$$

with
R₀ being the direct current resistance of the conductor at 20° C., in Ohm,
Yₛ being the skin effect factor, no units,
Yₚ being the proximity effect factor, no units,
α₂₀ being the coefficient of electrical resistivity, in K⁻¹ (i.e. per kelvin).

The calculation unit 120 is then configured to determine a power loss PₒL as a function of the electrical intensity I_{cond} calculated in the electrical conductor and the electrical resistance R_{c} of the electrical conductor that has been determined.

This power loss PₒL is in particular determined as follows:

$$PoL = Rc \times {I}_{cond}^{2}$$

The calculation unit 120 is configured to then determine the annual power losses induced by the heating of the electrical conductor 10. The quantity of emissions of carbon dioxide is then determined as a function of these annual power losses.

To determine the quantity of emissions of CO₂ as a function of these annual power losses, it is possible to use a scale factor multiplied by the annual power losses.

Generally speaking, the quantity of emissions of CO₂ as a function of these annual power losses can be determined using a relation defined by a distribution system operator.

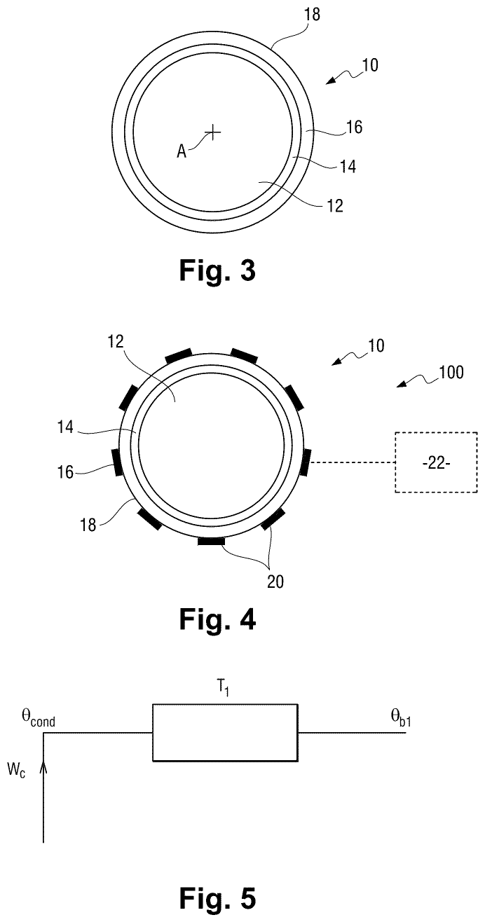

Referring to FIG. 3, an electrical cable 10 comprises an electrical conductor 12, a first layer of material 14 around the electrical conductor 12, and a second layer of material 16 around the first layer of material 14.

The electrical conductor 12 extends along a longitudinal axis A.

The first layer 14 of material and the second layer 16 of material extend along the longitudinal axis A around the electrical conductor 12.

The first layer 14 of material is for example a layer formed of an electrically insulating material. The first layer 14 of material can therefore be considered as an insulative layer.

The second layer 16 of material here forms an external layer of the electrical cable 10. The second layer 16 of material forms an external surface 18 of the electrical cable 10.

The second layer 16 of material is for example an external sheath.

More generally, the electrical cable 10 may include one or more layers of material surrounding the electrical conductor 12. The electrical cable 10 may notably comprise one or more of the following: a screen, a semiconductor layer, an insulative layer, an external sheath.

In a preferred configuration the electrical cable 10 comprises around the conductor, in order of disposition from the centre to the periphery: a semiconductor layer, an insulative layer, a screen and an external sheath. This configuration corresponds for example to an electrical cable configured for a medium-voltage (between 1 and 52 kV) network.

In one embodiment, the determination system 100 is preferably non-invasive and/or non-destructive. In other words, the temperature sensor(s) 20 are not disposed inside the electric cable.

In this embodiment, the calculation unit 120 comprises a determination unit 22 configured to determine the conductor temperature Θ_{cond} by means of a physical model notably using a peripheral temperature Θ_{b1} measured by said at least one temperature sensor 20 disposed on an external surface of the electric cable 10.

The determination of this conductor temperature Θ_{cond} by means of this physical model is described below in connection with FIGS. 4 to 12.

As depicted in FIG. 4, the determination system 100 comprises said at least one temperature sensor 20 and a determination unit 22.

The determination unit 22 belongs to the calculation unit 120.

The determination system 100 may comprise a plurality of temperature sensors 20 distributed around the longitudinal axis A in a same plane transverse to this longitudinal axis A. In the example of FIG. 4, the determination system 100 comprises 9 temperature sensors 20.

The temperature sensor(s) 20 are configured to measure a peripheral temperature Θ_{b1}. In this configuration in which the temperature sensors 20 are disposed at the external surface 18 of the electric cable, the peripheral temperature Θ_{b1} corresponds to the surface temperature of the electric cable 10.

The temperature sensor or sensors 20 are connected to the determination unit 22 in such a manner as to communicate the peripheral temperature Θ_{b1} to this determination unit 22.

The temperature sensors 20 are preferably equally distributed around the longitudinal axis A. The determination system 100 may provide one or more temperature sensors 20 further distributed along the longitudinal axis A in such a manner as to measure the peripheral temperature Θb1 at different locations along the electrical cable 10.

This determination unit 22 is configured to determine the conductor temperature Θ_{cond}, i.e. the temperature of the electrical conductor 12.

This determination is performed in a non-invasive and non-destructive manner. Thus, no component is inserted under the material layers or in the proximity of the electrical conductor 12 to determine its conductor temperature Θ_{cond}. Furthermore, no layer of material of the electric cable 10 is damaged or pierced to carry out this determination. No third-party component is integrated in the fabrication of the electric cable 10 such as an optical fibre or a sensor in one of the layers of the electric cable 10 or between these layers of material.

This enables determination of the conductor temperature in an existing electrical cable, for example already installed in situ, without having to damage it or insert any measuring tool.

It is considered here that the addition of measurement components or additional layers of material is not invasive or destructive.

The determination unit 22 uses a physical model enabling the conductor temperature Θ_{cond} to be determined as a function of the peripheral temperature Θ_{b1} measured by the temperature sensor(s) 20.

Referring to FIG. 5, the diffusion of heat through the electrical cable 10 is represented in diagrammatic form to depict the physical model utilised by the determination unit 22.

This physical model is based on the fact that the diffusion of heat through the layers of an electric cable follows a behaviour close to that of the circulation of a current in an electric circuit including an electric resistance.

The physical model therefore establishes a relation between the thermal resistance T1 of said at least one layer of material. The thermal resistance T₁ may correspond to the thermal resistance of one or more of the layers of material. In the example of FIG. 4, the thermal resistance T₁ represents the thermal resistance of the combination of the first layer 14 of material and the second layer 16 of material. In this physical model, the first layer 14 of material and the second 16 layer of material therefore form one and the same layer of material having a layer thermal resistance denoted T₁.

The passage of the current inside the conductor generates heating inducing a heat flux W_{c}.

In this physical model the voltage difference ΔU at the boundaries of an electrical resistance is close to a temperature difference ΔΘ between the internal and external surfaces of said at least one layer of material (i.e. at the boundaries of this layer of material).

In the application of the electrical cable 10, the temperatures at the boundaries of the thermal resistance T₁ are firstly the temperature of the conductor Θ_{cond} and secondly the peripheral temperature Θ_{b1}. Thus, the temperature difference ΔΘ is expressed as follows: ΔΘ=Θ_{cond}−Θ_{b1}.

In this physical model, a mathematical relation is established between the heat flux W_{c}, the thermal resistance T₁ and the temperature difference ΔΘ at the boundaries of this thermal resistance. This relation is as follows:

ΔΘ=T₁*W_{c}
with
ΔΘ is the temperature difference ΔΘ between the internal and external surfaces [000131]
of said at least one layer of material,
T₁ being the thermal resistance of said at least one layer of material,
We being the heat flux generated by the heating of the conductor.

The conductor temperature Θ_{cond} can therefore be expressed as follows:

$${θ}_{cond} = {θ}_{b 1} + {W}_{c} * {T}_{1}$$

with
Θ_{cond} being the conductor temperature,
Θ_{b1} being the peripheral temperature.

The thermal resistance T₁ of said at least one layer of material is determined as follows:

$${T}_{1} = \frac{{ρ}_{T}}{2 \times π} ln ( 1 + 2 * \frac{{t}_{1}}{{d}_{c}} )$$

with
ρ_{T} being the thermal conductivity of said at least one layer of material,
d_{c} being the internal diameter of said at least one layer of material,
t₁ being the thickness of said at least one layer of material.

The determination system 100 comprises at least one additional layer 24 of material and at least one additional temperature sensor 26.

This determination mode makes it possible to dispense with the measurement of the electrical intensity I_{cond} of the current flowing in the conductor.

Said at least one additional layer of material 24 is disposed around said at least one layer of material. The additional layer(s) 24 of material covers the at least one temperature sensor 22, as can be seen in FIG. 6.

This determination mode can be used for determining the conductor temperature Θ_{cond} according to different models of an electric cable 10. These different models may involve different assumptions (e.g. taking dielectric losses into account or not) or different configurations of the electric cable 10.

The determination unit 22 is configured to determine the conductor temperature Θ_{cond} according to one or more models, notably one or more of the models presented hereinafter.

Electrical Cable with No Screen and Dielectric Losses not Taken into Account

The determination unit 22 is configured to determine the conductor temperature Θ_{cond} according to first and second models respectively depicted in FIGS. 5 and 7.

In the first and second models, the dielectric losses in said at least one layer of material are not taken into account or are considered to be minimal.

In these first and second models, the electric cable 10 has no screen.

The first model applies to an electrical cable 10 comprising an electrical conductor 12 and one or more layers of material surrounding the electrical conductor 12. One or more temperature sensors 20 is or are disposed on the external surface 18 of said at least one layer of material.

The electric cable 10 in FIG. 4 is an example compatible with this first model.

The determination unit 100 comprises an additional structure illustrated in FIG. 6. Thus, the determination system 100 comprises at least one additional layer 24 of material and at least one additional temperature sensor 26.

Said at least one additional layer 24 of material has an additional thermal resistance T_{b}.

Said at least one additional layer 24 of material is, for example, at least one electrically insulating layer.

The material of said at least one additional layer 24 of material preferably has a thermal resistance between 0.001 m²·K/W and 0.1 m²·K/W. In this thermal resistance range, said at least one layer 24 of material makes it possible to avoid overheating of the conductor while allowing a temperature difference that is large enough to be measured.

Said at least one additional temperature sensor 26 makes it possible to measure an additional peripheral temperature Θ_{b2} at the level of the external surface 28 of said at least one additional layer 24 of material.

The determination system 100 may comprise a plurality of additional temperature sensors 26 distributed around the longitudinal axis A in a same plane transverse to the longitudinal axis A. In the example of FIG. 6, the determination system 100 comprises 9 additional temperature sensors 26.

The additional temperature sensor or sensors 26 are connected to the determination unit 22 in such a manner as to communicate the additional peripheral temperature Θ_{b2} to this determination unit 22.

The additional temperature sensors 26 are preferably equally distributed around the longitudinal axis A. The determination system 100 may provide one or more additional temperature sensors 26 further distributed along the longitudinal axis A in such a manner as to measure the additional peripheral temperature Θ_{b2} at different locations along the electrical cable 10.

Preferably, the number and/or the angular position and/or the longitudinal position of the temperature sensors 20 are respectively identical to the number and/or the angular position and/or the longitudinal position of the additional temperature sensors 26.

The electric cable 10 equipped with said at least one additional layer 24 of material and said at least one additional temperature sensor 26 is modelled by a second model in FIG. 7. Said at least one additional layer 24 of material is considered to be a resistance of value T_{b} in series with the resistance of value T₁ corresponding to said at least one layer of material.

The thermal flux W_{c} is expressed as follows:

$${W}_{c} = \frac{( {Θ}_{b 1} - {Θ}_{b 2} )}{{T}_{b}}$$

The conductor temperature can thus be expressed as follows:

$${θ}_{cond} = {θ}_{b 1} + \frac{( {θ}_{b 1} - {θ}_{b 2} )}{{T}_{b}} \times {T}_{1}$$

The determination of the conductor temperature Θ_{cond} can thus be determined without requiring the value of the intensity of the current circulating in the electrical conductor 12. This determination is made possible by the addition of an additional layer and an additional sensor.

Electrical Cable with No Screen and with Dielectric Losses Taken into Account

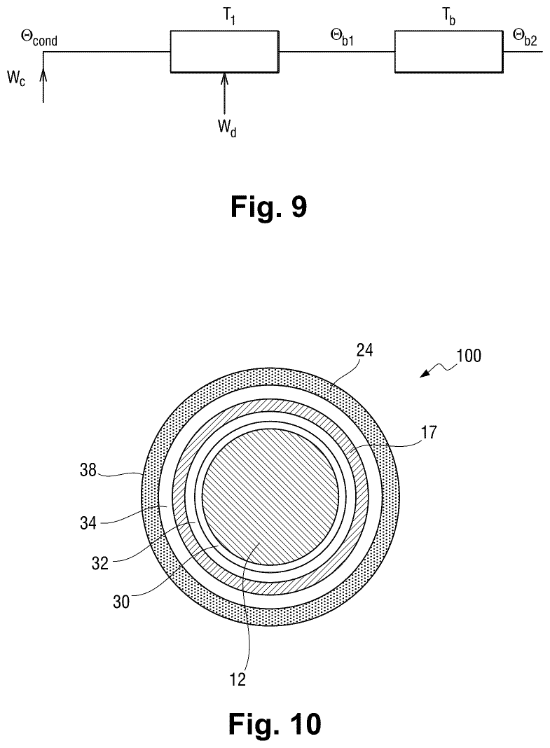

The determination unit 22 is configured to determine the conductor temperature Θ_{cond} according to third and fourth models respectively depicted in FIGS. 8 and 9.

.

In the third and fourth models, the dielectric losses in said at least one layer of material are taken into account.

In these third and fourth models, the electric cable 10 has no screen.

In these third and fourth models, the dielectric losses in said at least one layer of material are considered as a loss thermal flux W_{d} at the level of resistance of value T₁ corresponding to said at least one layer of material. This loss thermal flux W_{d} can be seen in FIGS. 8 and 9.

The third model applies to an electrical cable 10 comprising an electrical conductor 12 and one or more layers of material surrounding the electrical conductor 12. One or more temperature sensors 20 is or are disposed on the external surface 18 of said at least one layer of material.

The electric cable 10 according to FIG. 4 is an example compatible with this third model.

The determination unit 100 comprises an additional structure as illustrated in FIG. 6. Thus, the determination system 100 comprises at least one additional layer 24 of material and at least one additional temperature sensor 26.

The electric cable 10 equipped with said at least one additional layer 24 of material and said at least one additional temperature sensor 26 is modelled by a fourth model in FIG. 9.

Said at least one additional layer 24 of material is considered to be a resistance of value T_{b} in series with the resistance of value T₁ corresponding to said at least one layer of material.

The conductor temperature Θ_{cond} is expressed as follows:

$${θ}_{cond} = {θ}_{b 1} + {T}_{1} \times ( \frac{( {θ}_{b 1} - {θ}_{b 2} )}{{T}_{b}} - \frac{{W}_{d}}{2} )$$

The preceding equation is obtained by considering the following equations:

$${θ}_{cond} = {θ}_{b 1} + {T}_{1} \times ( {W}_{c} + \frac{{W}_{d}}{2} ) \\ and \\ {θ}_{b 1} = {θ}_{b 2} + {T}_{b} \times ( {W}_{d} + {W}_{c} )$$

The loss thermal flux W_{d} is determined as a function of the voltage applied to the electrical conductor 12, the frequency of the voltage applied to the electrical conductor 12 and the dielectric characteristics of said at least one layer of material.

Electrical Cable with Screen and with Dielectric Losses Taken into Account

The determination unit 22 is further configured to determine the conductor temperature Θ_{cond} in a configuration of the electrical cable 10 comprising a screen 17.

As depicted in FIG. 10, the electric cable 10 comprises an electrical conductor 12, one or more layers of material surrounding the electrical conductor 12, and a shield 17 surrounding said layers of material.

Said layers of material are for example a dielectric layer 30 surrounding the electrical conductor 12 and an insulative layer 32 disposed between the dielectric layer 30 and the screen 17.

The electrical cable 10 also comprises an external layer 34, for example an external sheath, defining an external surface 38 of the outer layer 34. The outer layer 34 may comprise a plurality of layers of material.

The outer layer 34 has a thermal resistance T₃.

One or more temperature sensors 20 is or are disposed on the external surface 38 of the external layer 34.

Losses in the screen 17 are modelled by a screen thermal flux Wₛ. These losses are due to heating by the Joule effect in the screen 17.

Determination of the screen thermal flux Wₛ requires an invasive measurement of the electrical cable 10. To avoid having to express the conductor temperature Θ_{cond} as a function of the thermal flux Wₛ, it is provided here to express the conductor temperature Θ_{cond} as a function of the intensity I_{cond} of the current circulating in the electrical conductor 12 and to utilise an additional structure comprising at least one additional layer 24 of material and at least one additional temperature sensor 26, as can be seen in FIG. 10.

Said at least one additional layer 24 of material has a thermal resistance T_{b}.

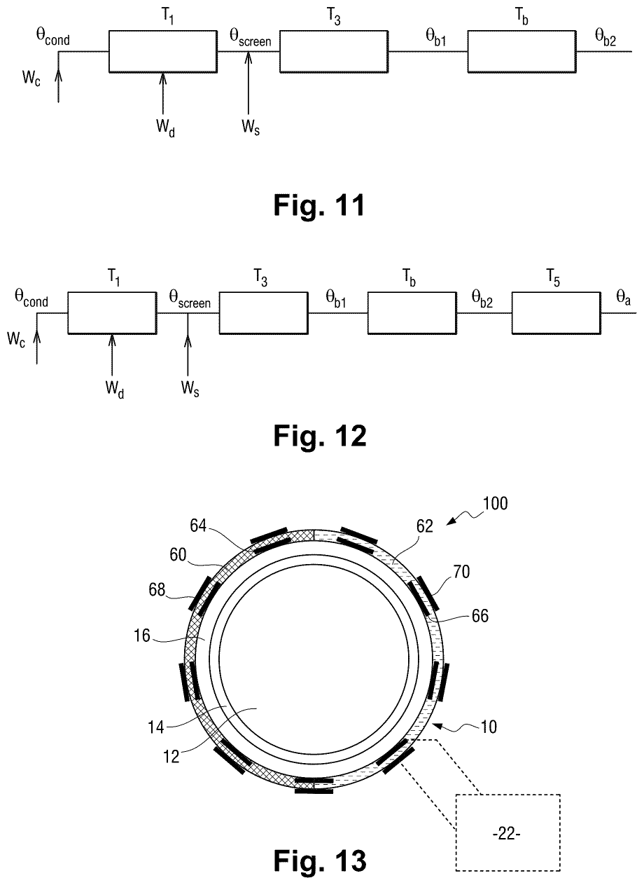

A fifth model is depicted in FIG. 11 taking into account the screen losses (thermal flux Wₛ) as well as the dielectric losses in said at least one layer (thermal flux W_{d}) and comprising three resistors in series to model the thermal resistances of said at least one layer of material (T₁), of the external sheath 34 (T₃) and of said at least one additional layer 24 of material (T_{b}).

In this fifth model, the conductor temperature Θ_{cond} is expressed as follows:

$${θ}_{cond} = \frac{{θ}_{b 1} + {T}_{3} \frac{{Δθ}_{b}}{{T}_{b}} + \frac{1}{2} {T}_{1} {W}_{d} - ( {R}_{0} {I}^{2} ) {T}_{1} ( 1 - 20 \times {α}_{20} )}{1 - {R}_{0} {I}_{cond}^{2} {α}_{20} {T}_{1}}$$

Θ_{b1} being the peripheral temperature measured by said at least one temperature sensor 20,
Θ_{b2} being the additional peripheral temperature measured by said at least one additional temperature sensor 26,
ΔΘ being the temperature difference Θ_{b1}−Θ_{b2},
T₁ being the thermal resistance of said at least one layer of material,
T_{b} being the thermal resistance of said at least one additional layer 24 of material,
T₃ being the thermal resistance of the external layer 34,
R₀ being the direct current resistance of the conductor at 20° C., expressed in Ohms,
Yₛ being the skin effect factor, no units,
Yₚ being the proximity effect factor, no units,
α₂₀ being the electrical resistivity coefficient, expressed in K⁻¹ (i.e. per kelvin).

The determination unit 22 is thus capable of determining the conductor temperature Θ_{cond} independently of the screen thermal flux Wₛ.

This expression of the conductor temperature Θ_{cond} is obtained considering that:

$${θ}_{cond} = {θ}_{surf} + n ( {W}_{c} + {W}_{s} + {W}_{d} ) {T}_{3} + ( {W}_{c} + \frac{{W}_{d}}{2} ) {T}_{1} \\ n ( {W}_{c} + {W}_{s} + {W}_{d} ) = \frac{{Δθ}_{b}}{{T}_{b}} \\ {θ}_{conductor} = {θ}_{b 1} + {T}_{3} \frac{{Δθ}_{b}}{{T}_{b}} + ( {W}_{c} + \frac{{W}_{d}}{2} ) {T}_{1}$$

with Θ_{surf} being the peripheral temperature at the level of the external surface of the electrical cable 10, and
n being the number of electrical conductors 12.

As described in detail above, the thermal resistance T₁ is determined as follows:

$${T}_{1} = \frac{{ρ}_{T}}{2 \times π} ln ( 1 + 2 * \frac{{t}_{1}}{{d}_{c}} )$$

The thermal resistance T₃ of the external layer 34 is determined as follows:

$${T}_{3} = \frac{{ρ}_{T}}{2 \times π} ln ( 1 + 2 * \frac{{t}_{3}}{{D}_{a}} )$$

with t₃ being the thickness of the external layer 34,
Dₐ being the inside diameter of the external layer 34.

In a sixth model depicted in FIG. 12, the determination unit is also configured to determine the conductor temperature Θ_{cond} in a manner independent of the surrounding environment, notably the temperature in that surrounding environment.

This sixth model applies to the same configuration of the electrical cable 10 as the fifth model. In other words, the sixth model applies to an electric cable of the type of the FIG. 10 type with an additional structure and a screen 17.

Depending on the surrounding environment, the heat will be evacuated less or more from the electric cable 10. The surrounding environment is modelled by a layer of material with a certain thermal resistance T₅ and a temperature θₐ corresponding to the ambient temperature of the surrounding environment.

The conductor temperature Θ_{cond} may be expressed as follows:

$${θ}_{cond} = {θ}_{a} + ( {W}_{c} + \frac{{W}_{d}}{2} ) {T}_{1} + n ( {W}_{c} + {W}_{d} + {W}_{s} ) {T}_{3} + n ( {W}_{c} + {W}_{d} + {W}_{s} ) {T}_{b} + n ( {W}_{c} + {W}_{d} + {W}_{s} ) {T}_{5}$$

The temperature difference Θ_{b1}−Θ_{b2} on either side of the additional layer 24 of material enables the conductor temperature Θ_{cond} to be expressed as follows:

$${θ}_{cond} = {θ}_{b 1} + ( {W}_{c} + \frac{{W}_{d}}{2} ) {T}_{1} + n ( {W}_{c} + {W}_{d} + {W}_{s} ) {T}_{b} \\ and \\ {θ}_{b 1} - {θ}_{b 2} = n ( {W}_{c} + {W}_{d} + {W}_{s} ) {T}_{b}$$

The conductor temperature Θ_{cond} can therefore be determined by the determination unit 22 independently of the surrounding environment.

Referring to FIGS. 13 to 19, the determination unit 22 is furthermore configured to determine the conductor temperature Θ_{cond} in a seventh model and an eighth model.

In the seventh and eighth models, the determination unit 22 is configured to determine the conductor temperature Θ_{cond} without necessitating determination of the thermal flux W_{c} generated by the circulation of an electric current in the electrical conductor.

For these seventh and eighth models, the determination system 100 is similar to that of FIG. 6 with the difference that said at least one additional layer 24 comprises a first 60 and a second 62 additional layer portions, as depicted in FIG. 13.

The first additional layer portion 60 has a first additional thermal resistance T_{b}. The second additional layer portion 62 has a second additional thermal resistance T′_{b}. The first additional thermal resistance T_{b} and the second additional thermal resistance T′_{b} are different.

This difference between the first additional thermal resistance T_{b} and the second additional thermal resistance T b can be obtained by utilisation of a different material and/or one or more different geometric characteristics that differ between the first additional layer portion 60 and the second additional layer portion 62. One example of a different geometric characteristic is a different thickness, taken along an axis perpendicular to the longitudinal axis of extension A of the electrical conductor.

This difference between the first additional thermal resistance T_{b} and the second additional thermal resistance T_{b} enables construction of two different equations having for unknown the conductor temperature Θ_{cond}. It is therefore possible to dispense with knowing the thermal flux W_{c}.

A plurality of first temperature sensors 64 are disposed on the external surface 18 of said at least one layer of material, between said external surface 18 and the first additional layer portion 60. The plurality of first temperature sensors 64 enable measurement of a first peripheral temperature Θ_{b1} at the level of the external surface 18 of said at least one layer of material.

A plurality of second temperature sensors 66 are disposed on the external surface 18 of said at least one layer of material, between said external surface 18 and the second additional layer portion 62. The plurality of second temperature sensors 66 enable measurement of a second peripheral temperature Θ′_{b1} at the level of the external surface 18 of said at least one layer of material.

A plurality of first additional temperature sensors 68 are disposed on an external surface of the first additional layer portion 60 to measure a first additional peripheral temperature Θ_{b2}.

A plurality of second additional temperature sensors are disposed on an external surface of the second additional layer portion to measure a second additional peripheral temperature Θ_{b2}.

The determination unit 22 is configured to determine the conductor temperature Θ_{cond} as a further function of the first additional thermal resistance T_{b} and the second additional thermal resistance T′_{b} and the first additional peripheral temperature Θ_{b2} and the second additional peripheral temperature Θ′_{b2}.

Electrical Cable with No Screen without Dielectric Losses Taken into Account

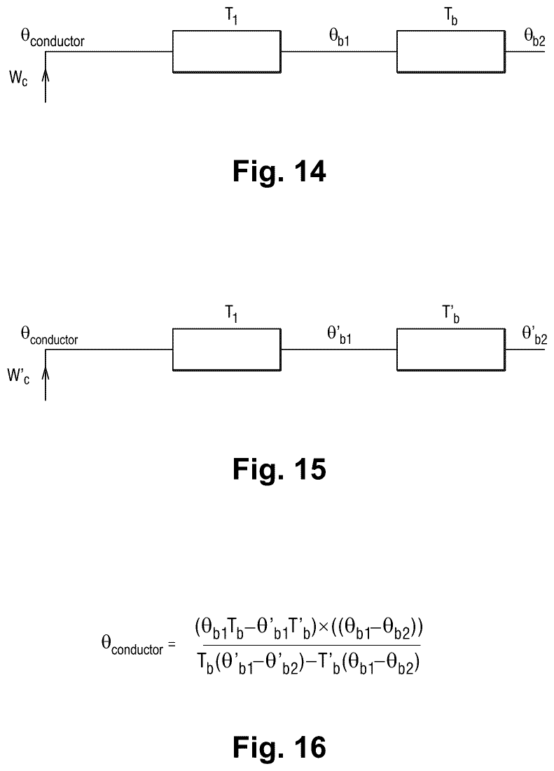

The seventh model is depicted in FIGS. 14 to 16.

In the seventh model, the conductor temperature Θ_{cond} is expressed as follows:

$${θ}_{conductor} = \frac{( {θ}_{b 1} {T}_{b} - {θ}_{b 1}^{′} {T}_{b}^{′} ) \times ( ( {θ}_{b 1} - {θ}_{b 2} ) )}{{T}_{b} ( {θ}_{b 1}^{′} - {θ}_{b 2}^{′} ) - {T}_{b}^{′} ( {θ}_{b 1} - {θ}_{b 2} )}$$

This equation is obtained via the following expansions:

$$\frac{( {θ}_{conductor} - {θ}_{b 1} )}{{T}_{1}} = \frac{( {θ}_{b 1} - {θ}_{b 2} )}{{T}_{b}} \\ \frac{( {θ}_{conductor} - {θ}_{b 1}^{′} )}{{T}_{1}} = \frac{( {θ}_{b 1}^{′} - {θ}_{b 2}^{′} )}{{T}_{b}^{′}} \\ ( {θ}_{conductor} - {θ}_{b 1} ) \frac{{T}_{b}}{( {θ}_{b 1} - {θ}_{b 2} )} = ( {θ}_{conductor} - {θ}_{b 1}^{′} ) \frac{{T}_{b}^{′}}{( {θ}_{b 1}^{′} - {θ}_{b 2}^{′} )} \\ {θ}_{conductor} = \frac{{θ}_{b 1} {T}_{b} - {θ}_{b 1}^{′} {T}_{b}^{′}}{( {θ}_{b 1}^{′} - {θ}_{b 2}^{′} )} / ( \frac{{T}_{b}}{( {θ}_{b 1} - {θ}_{b 2} )} - \frac{{T}_{b}^{′}}{( {θ}_{b 1}^{′} - {θ}_{b 2}^{′} )} )$$

Electrical Cable with No Screen with Dielectric Losses Taken into Account

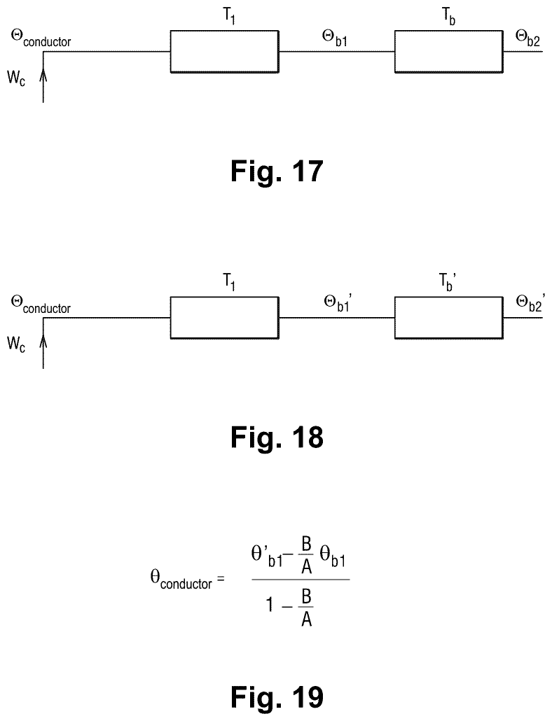

The eighth model is depicted in FIGS. 17 to 19.

In the eighth model, the conductor temperature Θ_{cond} is expressed as follows:

$${θ}_{conductor} = \frac{{θ}_{b 1}^{′} - \frac{B}{A} {θ}_{b 1}}{1 - \frac{B}{A}}$$

This equation is obtained via the following expansions:

$${θ}_{conductor} = {θ}_{b 1} + {T}_{1} \times ( \frac{( {θ}_{b 1} - {θ}_{b 2} )}{{T}_{b}} - \frac{{W}_{d}}{2} ) \\ {θ}_{conductor} = {θ}_{b 1}^{′} + {T}_{1} \times ( \frac{( {θ}_{b 1}^{′} - {θ}_{b 2}^{′} )}{{T}_{b}^{′}} - \frac{{W}_{d}}{2} ) \\ A = ( \frac{( {θ}_{b 1} - {θ}_{b 2} )}{{T}_{b}} - \frac{{W}_{d}}{2} ) \\ B = ( \frac{( {θ}_{b 1}^{′} - {θ}_{b 2}^{′} )}{{T}_{b}^{′}} - \frac{{W}_{d}}{2} )$$

Θ_{b1}′ being the first peripheral temperature at the level of the external surface 18 of said at least one layer of material,
Θ_{b2}′ being the second additional peripheral temperature, T_{b}′ being the second additional thermal resistance.

Naturally, the invention is not limited to the embodiments described, but includes any variant entering into the scope of the invention such as defined by the claims.

In particular, it is understood that, while it is advantageous to be able to calculate the intensity of the current flowing in the conductor 12 from the temperature measurement (without the need for a dedicated current sensor), this calculation is not necessary to determine the emissions of carbon dioxide. The emissions of carbon dioxide can be determined solely from the temperature measurements by calculating the thermal power (or thermal flux) produced by the Joule effect by the current circulating in the conductor 12. It is indicated that tables relating emissions of carbon dioxide and thermal power are used for this purpose, but it is possible alternatively to use a numerical model, for example developed empirically.

The system may include one or more temperature sensors.

1. A system for determination of a quantity of emissions of carbon dioxide resulting from the heating of an electrical conductor of an electrical cable by the Joule effect, said determination system comprising:
an electrical cable comprising at least one electrical conductor and at least one layer of material surrounding said at least one conductor,
a measuring unit associated with the electrical cable, said measuring unit comprising at least one temperature sensor,
a calculation unit being configured to communicate information with the measurement unit, the calculation unit being configured to determine the conductor temperature Θ_{cond} by means of said at least one temperature sensor, said calculation unit being further configured to determine an quantity of emissions of carbon dioxide resulting from the heating of the electrical conductor by the Joule effect as a function of the conductor temperature Θ_{cond}.

2. The system according to claim 1, wherein the calculation unit is configured to determine an electrical intensity I_{cond} of the electrical conductor as a function of the conductor temperature Θ_{cond}.

3. The system according to claim 1, wherein the calculation unit is configured to determine an electrical resistance R_{c} of the electrical conductor as a function of the conductor temperature Θ_{cond}.

4. The system according to claim 3, wherein the calculation unit is configured to determine a power loss PₒL as a function of the electrical intensity I_{cond} in the electrical conductor and the electrical resistance R_{c} of the electrical conductor, the calculation unit being configured to determine the amount of emissions of carbon dioxide as a function of said power loss PₒL.

5. The system according to claim 1, further comprising a measurement module mounted on the electrical cable, said measurement module comprising at least said at least one temperature sensor.

6. The system according to claim 5, wherein the measurement module further comprises the calculation unit.

7. The system according to claim 5, wherein the measurement module is configured to be removably mounted on the electrical cable.

8. The system according to claim 7, wherein the measurement module comprises a device for fixing it to the electrical cable.

9. The system according to claim 1, wherein said at least one temperature sensor comprises a temperature sensor that is disposed on an external surface of said at least one layer of material to measure a peripheral temperature Θ_{b1} at the level of the external surface of said at least one layer of material, the calculation unit being configured to determine the conductor temperature Θ_{cond} as a function of the peripheral temperature Θ_{b1}.

10. The system according to claim 9, further comprising:
an additional layer of material disposed around said at least one layer of material and covering said at least one temperature sensor,
at least one additional temperature sensor disposed on an external surface of said layer of additional material to measure an additional peripheral temperature Θ_{b2} at the level of the external surface of said at least one additional layer of material.

11. The system according to claim 1, wherein the calculation unit is configured to determine the conductor temperature Θ_{cond} as a function of the peripheral temperature Θ_{b1} and the additional peripheral temperature Θ_{b2}.

12. The system according to claim 1, wherein the calculation unit is configured to determine the electrical intensity I_{cond} as a function of the conductor temperature Θ_{cond} and the conductor geometry.

13. The system according to claim 10, wherein said at least one additional layer of material comprises:
a first additional layer portion having a first additional thermal resistance T_{b}, and
a second additional layer portion having a second additional thermal resistance T′_{b}, the first T_{b} and second T′_{b} additional thermal resistances being different,
and in which said at least one additional temperature sensor comprises:
at least one first additional temperature sensor disposed on an external surface of the first additional layer portion to measure a first additional peripheral temperature Θ_{b2},
at least one second additional temperature sensor disposed on an external surface of the second additional layer portion to measure a second additional peripheral temperature Θ′_{b2},
the determination unit being configured to determine the conductor temperature Θ_{cond} as a function of the first T_{b} and the second T′_{b} additional thermal resistances as well as the first Θ_{b2} and the second Θ′_{b2} additional peripheral temperatures.

14. The system according to claim 1, wherein said at least one first and at least one second additional layers are disposed on different angular sectors around the electrical conductor.

15. The system according to claim 1, wherein said at least one temperature sensor comprises:
at least one first sensor disposed on the external surface of said at least one layer of material, between said external surface and the first additional layer portion, to measure a peripheral temperature Θ_{b1} at the level of the external surface of said at least one layer of material,
at least one second sensor disposed on the external surface of said at least one layer of material, between said external surface and the second additional layer portion, to measure a peripheral temperature Θ′_{b1} at the level of the external surface of said at least one layer of material.

16. The system according to claim 1, wherein the determination unit is configured to determine the conductor temperature Θ_{cond} on the basis of the following equation:
$${θ}_{conductor} = \frac{( {θ}_{b 1} {T}_{b} - {θ}_{b 1}^{′} {T}_{b}^{′} ) \times ( ( {θ}_{b 1} - {θ}_{b 2} ) )}{{T}_{b} ( {θ}_{b 1}^{′} - {θ}_{b 2}^{′} ) - {T}_{b}^{′} ( {θ}_{b 1} - {θ}_{b 2} )}$$
T_{b} being the first additional thermal resistance of the first additional layer of material,
T′_{b} being the second additional thermal resistance of the second additional layer of material,
Θ_{b1} is the peripheral temperature measured by said at least one first temperature sensor,
Θ_{b2} is the additional peripheral temperature measured by said at least one first additional temperature sensor,
Θ′_{b1} is the peripheral temperature measured by said at least one second temperature sensor,
Θ′_{b2} is the additional peripheral temperature measured by said at least one second additional temperature sensor.

17. The system according to claim 12, wherein the first and second additional layer portions are made of a different material.

18. The system according to claim 12, wherein the first and second additional layer portions have a different thickness, perpendicular to a longitudinal axis along which the electrical cable extends.
